(19) United States
(12) Patent Application Publication
(10) Pub. No.: US 2026/0338712 A1
(43) Pub. Date: Oct. 29, 2026

(54) RADIATION IMAGING APPARATUS, RADIATION IMAGING SYSTEM, METHOD OF OPERATING RADIATION IMAGING APPARATUS, AND COMPUTER READABLE STORAGE MEDIUM

(71) Applicant: CANON KABUSHIKI KAISHA, Tokyo (JP)

(72) Inventor: KAI SUZUKI, Kanagawa (JP)

(21) Appl. No.: 19/705,150

(22) Filed: Jun. 11, 2026

Related U.S. Application Data
(63) Continuation of application No. PCT/JP2024/042625, filed on Dec. 3, 2024.

(30) Foreign Application Priority Data
Dec. 14, 2023 (JP) 2023-210972

(51) Int. Cl. G01N 23/04; G01P 1/07; G01P 13/00

(52) U.S. Cl. CPC G01N 23/04; G01P 1/07; G01P 13/00; G01N 2223/306; G01N 2223/408

(57) ABSTRACT

A radiation imaging apparatus is provided that includes a first measuring unit that measures motion information of the radiation imaging apparatus and a second measuring unit that has a dynamic range smaller than a dynamic range of the first measuring unit and measures motion information of the radiation imaging apparatus, wherein application of an impact to the radiation imaging apparatus is determined using the first measuring unit, and information indicating an orientation of the radiation imaging apparatus is calculated using the second measuring unit.

### CROSS-REFERENCE TO RELATED APPLICATIONS

This application is a Continuation of International Patent Application No. PCT/JP2024/042625, filed Dec. 3, 2024, which claims the benefit of Japanese Patent Application No. 2023-210972, filed Dec. 14, 2023, both of which are hereby incorporated by reference herein in their entirety.

### BACKGROUND

### Field of the Technology

The present disclosure relates to a radiation imaging apparatus, a radiation imaging system, a method of operating a radiation imaging apparatus, and a computer readable storage medium.

### Description of the Related Art

Currently, flat panel detectors (FPDs) made of semiconductor materials are widely used as radiation detectors for medical imaging diagnosis and nondestructive testing using radiation such as X-rays. Also, a radiation imaging system in which such a radiation detector and a radiation generating apparatus that generates radiation are combined is used.

Products that incorporate a gravity sensor into such a radiation imaging system have been put to practical use. As a function of a radiation imaging system including a gravity sensor, a function of deriving an orientation of a radiation generating apparatus and a radiation detector and displaying the orientation on a display unit or the like has been put into practical use. By the function, the radiation imaging system can assist alignment between an irradiation field plane of radiation emitted from the radiation generating apparatus and an incident surface of the radiation detector and can detect that an impact has been applied to the radiation detector.

There is a radiation imaging system in which a gravity sensor is provided inside the radiation detector and an orientation state of the radiation detector is calculated from an output value of the gravity sensor as one example of alignment between a radiation generating apparatus and a radiation detector.

For example, in a technique described in Japanese Patent Laid-Open No. 2021-45647, alignment between a radiation imaging apparatus and a radiation generating apparatus is performed by using a first tilt angle detecting unit that detects a tilt angle of the radiation imaging apparatus and a second tilt angle detecting unit that detects a tilt angle of a radiation source of a radiation generating apparatus. Also, as one example of impact detection, Japanese Patent No. 7207482 discloses a method of detecting an impact by arranging an acceleration sensor at a center of a radiation imaging apparatus.

In the technique described in Japanese Patent Laid-Open No. 2021-45647, a tilt angle of a radiation imaging apparatus can be detected by using a gravity sensor, and in the technique described in Japanese Patent No. 7207482, an impact on a radiation imaging apparatus can be detected by using an acceleration sensor (gravity sensor). However, although it is also a gravity sensor, the gravity sensor for orientation detection used in Japanese Patent Laid-Open No. 2021-45647 is generally a sensor having a small dynamic range, and for a large signal such as an impact, the sensor is saturated and cannot detect a peak. On the other hand, the gravity sensor for impact detection used in Japanese Patent No. 7207482 generally has a large dynamic range but has low sensitivity and a large amount of noise, and is not suitable for use in orientation detection. Therefore, in the techniques described in Japanese Patent Laid-Open No. 2021-45647 and Japanese Patent No. 7207482, it has been difficult to achieve both orientation detection and impact detection of a radiation imaging apparatus.

### SUMMARY

According to an aspect of the present disclosure, there is provided a radiation imaging apparatus having improved functions of detecting an orientation of the radiation imaging apparatus and detecting an impact on the radiation imaging apparatus.

A radiation imaging apparatus according to one implementation of the present disclosure includes a first measuring unit that measures motion information of the radiation imaging apparatus and a second measuring unit that has a dynamic range smaller than a dynamic range of the first measuring unit and measures motion information of the radiation imaging apparatus, wherein application of an impact to the radiation imaging apparatus is determined using the first measuring unit, and information indicating an orientation of the radiation imaging apparatus is calculated using the second measuring unit.

Features of the present disclosure will become apparent from the following description of embodiments with reference to the attached drawings.

### BRIEF DESCRIPTION OF THE DRAWINGS

FIG. 1 is a diagram for illustrating an example of a schematic configuration of a radiation imaging system according to a first embodiment.

FIG. 2A is a diagram for describing an orientation calculation operation using a measuring unit according to the first embodiment.

FIG. 2B is a diagram for describing an orientation calculation operation using a measuring unit according to the first embodiment.

FIG. 3A is a diagram for illustrating an example of a display during alignment according to the first embodiment.

FIG. 3B is a diagram for illustrating an example of a display during alignment according to the first embodiment.

FIG. 4 is a flowchart illustrating a series of operations according to the first embodiment.

FIG. 5 is a flowchart illustrating a series of operations according to a second embodiment.

FIG. 6 is a diagram for illustrating an example of a schematic configuration of a radiation imaging system according to a third embodiment.

FIG. 7 is a flowchart illustrating a series of operations according to the third embodiment.

### DESCRIPTION OF THE EMBODIMENTS

Embodiments of the radiation imaging apparatus and a radiation imaging system of the present disclosure are described below with reference to drawings. The following embodiments do not limit the disclosure according to the claims. Although a plurality of features are described in the embodiments, not all of the plurality of features may be essential to the disclosure, and the plurality of features may be combined freely. Further, in the drawings, the same reference numerals are assigned to the same or similar configurations, and redundant description is omitted.

The following describes a radiation imaging system that uses X-rays as an example of radiation. However, the radiation may be X-rays or other radiation. In the following embodiments, the term radiation can include, for example, electromagnetic radiation such as X-rays and γ-rays, and particle radiation such as α-rays, β-rays, particle beams, proton beams, heavy ion beams, and meson beams.

### First Embodiment

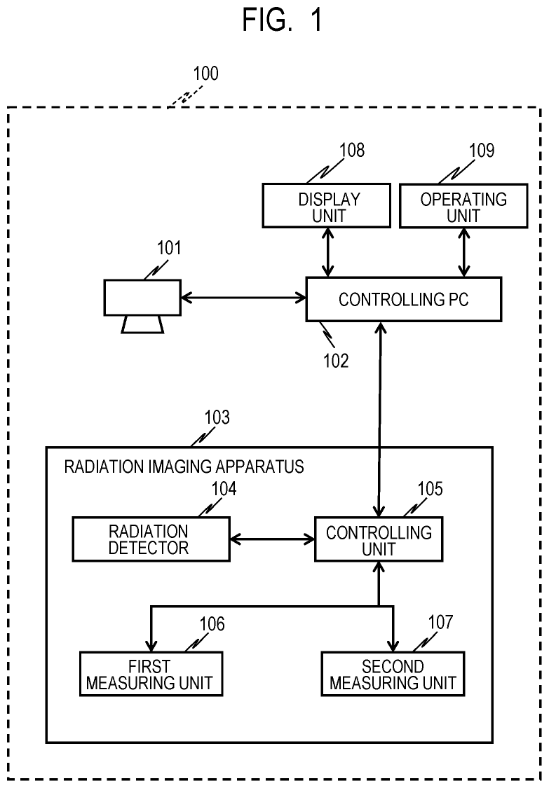

Referring to FIG. 1 to FIG. 5, a radiation imaging system, a radiation imaging apparatus and a method of operating a radiation imaging apparatus according to a first embodiment of the present disclosure are described below. In the first embodiment, a method for achieving both orientation detection and impact detection is described by taking, as an example, a radiation imaging apparatus including two acceleration sensors having different dynamic ranges as measuring units.

FIG. 1 illustrates an example of the schematic configuration of the radiation imaging system according to the first embodiment. A radiation imaging system 100 includes a radiation generating apparatus 101, a controlling PC 102, and a radiation imaging apparatus 103.

The radiation generating apparatus 101 includes, for example, a radiation generator such as a radiation tube, a collimator and a collimator lamp. The radiation generating apparatus 101 emits radiation under control of the controlling PC 102.

The radiation imaging apparatus 103 may be configured by any radiation detector that detects incident radiation and outputs a corresponding signal and may be configured by, for example, a flat panel detector (FPD) or the like. The radiation imaging apparatus 103 may be an indirect conversion type detector that converts radiation into visible light using a scintillator or the like and converts the visible light into an electrical signal using an optical sensor or the like, or may be a direct conversion type detector that directly converts incident radiation into an electrical signal. The radiation imaging apparatus 103 can detect incident radiation and send a signal corresponding to the detected radiation to the controlling PC 102.

The controlling PC 102 is connected to the radiation generating apparatus 101 and the radiation imaging apparatus 103 and can control them. The controlling PC 102 can also perform image processing on radiation images sent from the radiation imaging apparatus 103. Further, the controlling PC 102 is connected to a display unit 108 and an operating unit 109. The controlling PC 102 can serve as an example of a display controlling unit that controls display of the display unit 108.

The operating unit 109 is equipped with an input device such as a mouse and a keyboard, which can be operated by an operator to input an instruction to the controlling PC 102. The display unit 108 includes, for example, any monitor, and is capable of displaying information and an image output from the controlling PC 102 and information input via the operating unit 109.

In FIG. 1, a case is illustrated in which the radiation generating apparatus 101 and the radiation imaging apparatus 103 are connected via the controlling PC 102. The connection between them may take a plurality of forms, including direct cable connection, connection via a switching hub, and wireless connection. The radiation generating apparatus 101 may be installed in a room, or it may be mobile, such as a medical cart. Further, with respect to an imaging method, in addition to synchronous imaging in which the radiation generating apparatus 101 and the radiation imaging apparatus 103 are connected and imaging is performed in synchronization therebetween, asynchronous imaging in which imaging is performed by detecting irradiation of radiation without using a synchronization signal is also possible.

In the first embodiment, the controlling PC 102, operating unit 109, display unit 108, etc. are configured by separate apparatuses, but they may be configured by a single unit. For example, the operating unit 109 and the display unit 108 may be configured by a touch panel display.

The controlling PC 102 may be configured using a general-purpose computer including a processor and a memory or the like, or may be configured as a dedicated computer for the radiation imaging system 100. The controlling PC 102 may be, for example, a personal computer, a desktop PC, a laptop PC, or a tablet PC (portable information terminal). The processor may be a central processing unit (CPU). The processor may be, for example, a micro processing unit (MPU), graphical processing unit (GPU) or field-programmable gate array (FPGA).

Each function of the controlling PC 102 may be realized by a processor such as CPU or MPU executing a software module stored in a memory or other storage device. It should be noted that the processor may be a GPU, an FPGA or the like, for example. Each function may be configured by a circuit or other device that performs a specific function, such as an ASIC.

Next, operations of respective units when imaging is performed using the radiation imaging system 100 will be described. An operator (user) of the radiation imaging system 100 turns on power of respective apparatuses to bring the apparatuses into an imaging-ready state and performs position adjustment such as arrangement and an irradiation region of radiation of the radiation generating apparatus 101, and arrangement of the radiation imaging apparatus 103. For position adjustment, orientation information of the radiation imaging apparatus 103 and relative position and angle information of the radiation imaging apparatus 103 with respect to the radiation generating apparatus 101 can be used as auxiliary information, and such information can be displayed on the display unit 108.

The radiation generating apparatus 101 controls a radiation source to emit radiation in response to a radiation irradiation switch, not illustrated, being pressed or a radiation irradiation instruction being input from the controlling PC 102 in response to a user instruction via the operating unit 109. Radiation emitted from the radiation source passes through a subject (not illustrated) and enters the radiation imaging apparatus 103. The radiation imaging apparatus 103 generates an image corresponding to incident radiation and transmits the image to the controlling PC 102. The controlling PC 102 causes the received images to be displayed on a display unit such as the display unit 108 so that the user can check the displayed images and decide whether re-imaging is necessary or not, for example. If the user determines that the displayed image is normal, he/she prepares to image another subject using the same procedure.

The radiation imaging apparatus 103 may be configured by using a radiation detector such as an FPD. The radiation imaging apparatus 103 includes a radiation detector 104, a controlling unit 105, a first measuring unit 106, and a second measuring unit 107.

The radiation detector 104 includes pixels arranged in a two-dimensional matrix, switch elements for respective pixels, a drive circuit that controls the switch elements, and a readout circuit that reads charge from the pixels (all not illustrated). The drive circuit and the readout circuit are connected to the controlling unit 105 and are controlled by the controlling unit 105. The drive circuit is connected to switch elements of respective pixels via drive lines, and controls the switch elements of the respective pixels under control of the controlling unit 105. The readout circuit is connected to respective pixels via readout lines, and reads signals from the respective pixels under control of the controlling unit 105. The read-out image signal is sent to the controlling unit 105, and the controlling unit 105 generates a radiation image based on the image signal. The generated radiation image is sent to the controlling PC 102.

Further, the controlling unit 105 calculates orientation information of the radiation imaging apparatus 103 based on an output of the second measuring unit 107. Further, the controlling unit 105 determines an impact based on an output of the first measuring unit 106. The controlling unit 105 can be configured using any processor. Further, each function of the controlling unit 105 may be realized by a processor such as CPU or MPU executing a software module stored in a memory or other storage device. It should be noted that the processor may be a GPU, an FPGA or the like, for example. Each function may be configured by a circuit or other device that performs a specific function, such as an ASIC.

The first measuring unit 106 and the second measuring unit 107 are used, during position adjustment of the radiation imaging apparatus 103, for output of orientation information of the radiation imaging apparatus 103 and detection of an impact. In the first embodiment, an example is described in which three-axis acceleration sensors are used as the first measuring unit 106 and the second measuring unit 107. The acceleration sensor is merely an example, and a three-axis gyro sensor that obtains angular velocity information, a six-axis inertial measurement unit (6-axis IMU) including an acceleration sensor and a gyro sensor, or a nine-axis IMU obtained by adding a geomagnetic sensor to the six-axis IMU may be used.

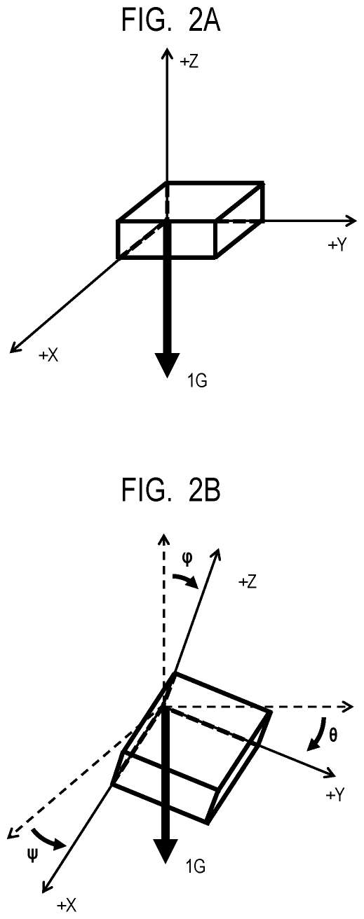

First, a method for obtaining orientation information (angle) of the radiation imaging apparatus 103 based on gravitational acceleration using a three-axis acceleration sensor will be described. As illustrated in FIG. 2A, the three-axis acceleration sensor outputs acceleration in respective axis directions in a three-axis orthogonal coordinate system (X-axis, Y-axis and Z-axis). The X and Y axes of the acceleration sensor are perpendicular to each other in a plane horizontal to the ground, and the Z axis is an angle perpendicular to the ground, with the X, Y, and Z axes being perpendicular to one another. In this case, gravitational acceleration is not applied in the x- and y-directions of the acceleration sensor, but only in the z-direction. Therefore, the output of the acceleration sensor is ax=ay=0 G and az=+1 G. Here, as illustrated in FIG. 2B, an angle between the X-axis of the acceleration sensor and the ground is defined as, an angle between the Y-axis of the acceleration sensor and the ground is defined as θ, and an angle between a gravity direction and the Z-axis of the acceleration sensor is defined as q. In this case, by using outputs ax, ay and az of the acceleration sensor, the following Equations 1 to 3 are satisfied, and an orientation of the radiation imaging apparatus 103 can be represented based on the outputs of the acceleration sensor.

$$θ = {tan}^{- 1} ( \frac{ax}{\sqrt{{ay}^{2} + {az}^{2}}} ) \qquad Equation 1$$

$$ψ = {tan}^{- 1} ( \frac{ay}{\sqrt{{ax}^{2} + {az}^{2}}} ) \qquad Equation 2$$

$$ϕ = {tan}^{- 1} ( \frac{\sqrt{{ax}^{2} + {ay}^{2}}}{az} ) \qquad Equation 3$$

Further, after appropriate initial values are provided, by time-integrating an output a of the acceleration sensor for each of the X-axis, Y-axis and Z-axis, a movement velocity v can be calculated as in the following Equation 4, and by time-integrating the movement velocity v, a position d can be calculated as in the following Equation 5.

$$v = v ( 0 ) + {\sum }_{k = 1}^{n} a ( k Δ t ) Δ t \qquad Equation 4$$

$$d = d ( 0 ) + {\sum }_{k = 1}^{n} v ( k Δ t ) Δ t \qquad Equation 5$$

The appropriate initial values referred to herein mean reference position coordinates for performing integration calculation. As a method for setting the reference position coordinates, there may be employed a method of setting, as a reference point, relative position coordinates between the radiation imaging apparatus 103 before movement and the radiation generating apparatus 101, relative position coordinates from any object, or coordinates of a specific location in an examination room.

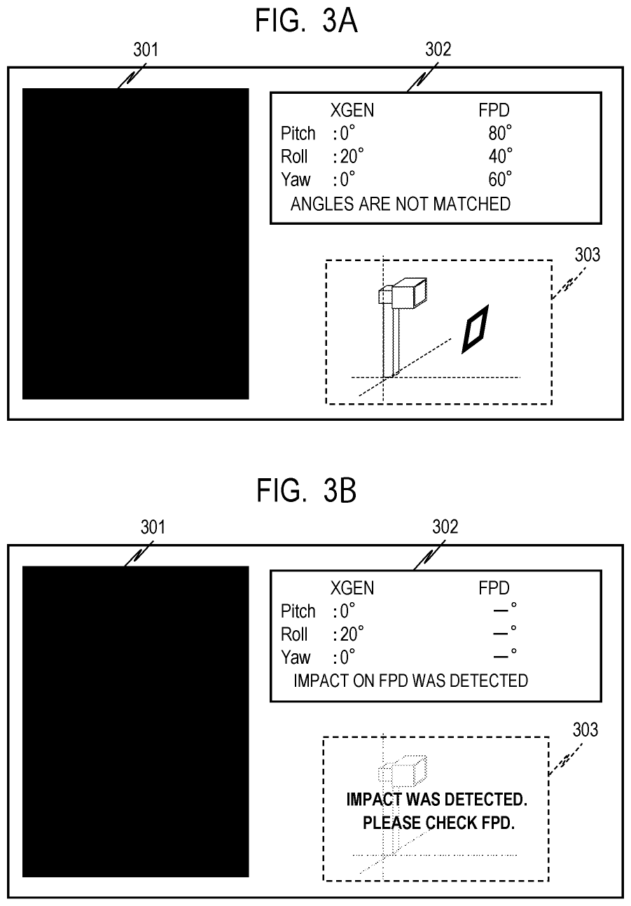

Here, FIG. 3A and FIG. 3B illustrate an example of a display of the display unit 108 during alignment. The display screens illustrated in FIG. 3A and FIG. 3B have an image display region 301, an orientation information display region 302, and a model display region 303. In the image display region 301, an image that was taken can be displayed. In the orientation information display region 302, angle information of the radiation generating apparatus 101 and the radiation imaging apparatus 103 can be displayed as numerical values. In the orientation information display region 302, position information of the radiation generating apparatus 101 and the radiation imaging apparatus 103 can also be displayed as numerical values. Further, in the model display region 303, a relative positional relationship between the radiation imaging apparatus 103 and the radiation generating apparatus 101 can be modeled and displayed so as to be visually easy to understand.

Further, depending on an imaging technique, when a positional relationship between the radiation imaging apparatus 103 and the radiation generating apparatus 101 becomes that has a preset angle or position, the controlling PC 102 can cause the display unit 108 to display a notification indicating that alignment is completed on the display unit 108. On the other hand, when a positional relationship between the radiation imaging apparatus 103 and the radiation generating apparatus 101 is not that has a preset angle or position, the controlling PC 102 can cause a notification indicating, for example, a mismatch in angle or the like to be displayed on the display unit 108, as illustrated in FIG. 3A. Further, the controlling PC 102 can cause a notification indicating that an impact has been detected to be displayed on the display unit 108, for example, as illustrated in FIG. 3B. The controlling PC 102 can also notify the user of the notification by sound or vibration.

Thus, the angle information and position information of the radiation imaging apparatus 103 allow the user to know the current orientation information of the radiation imaging apparatus 103. Therefore, by presenting angle information and position information of the radiation imaging apparatus 103, alignment between the radiation imaging apparatus 103 and the radiation generating apparatus 101 can be assisted.

On the other hand, during position adjustment, the radiation imaging apparatus 103 may be accidentally caused to collide with surrounding equipment or to be dropped. When angle or position of the radiation imaging apparatus 103 is finely adjusted, the radiation imaging apparatus 103 is not largely moved, and an output of an acceleration sensor becomes 1 G, which is a square root of a sum of squares of three-axis outputs and is approximately equal to gravitational acceleration. On the other hand, when the radiation imaging apparatus 103 is caused to collide with another apparatus or is dropped, an output of an acceleration sensor may greatly exceed gravitational acceleration of 1 G and be on an order of tens to hundreds of G.

In asynchronous imaging, when an impact is applied to the radiation imaging apparatus 103 at a timing of waiting for irradiation of radiation, there is a possibility that the impact is erroneously recognized as irradiation of radiation and an image is obtained even in a state in which radiation is not actually emitted. Also in synchronous imaging, there is a possibility that an impact is erroneously recognized as a radiation exposure permission signal, or an image artifact is generated due to the impact. Further, when a stronger impact is applied, the radiation imaging apparatus 103 may fail. In such a case, if an impact applied to the radiation imaging apparatus 103 can be known, it can assist, for example, in distinguishing between irradiation of radiation and the impact, preventing erroneous operation, identifying a cause of occurrence of an image artifact, and determining whether execution of self-diagnosis as to whether the radiation imaging apparatus 103 has failed is necessary.

For example, with respect to whether to perform self-diagnosis upon application of an impact, an operation may be employed in which, when the impact is equal to or greater than a preset threshold, the self-diagnosis is automatically executed, and when the impact is less than the threshold, a user is allowed to select whether to execute the self-diagnosis. Further, an impact may be detected during imaging preparation (alignment), and when the radiation imaging apparatus 103 makes transition to an imaging operation in a state before the radiation imaging apparatus 103 is set to a preset angle or position, a warning may be issued to notify a user. For example, it is possible to notify the user by displaying a message indicating that an impact has been detected on the display unit 108, as illustrated in the model display region 303 in FIG. 3B, or by notifying the user through any means, such as sound or vibration. Furthermore, by stopping the imaging operation of the radiation imaging apparatus 103 in response to the detection of an impact, it is possible to prevent erroneous operation.

Therefore, in the first embodiment, an acceleration sensor with a signal dynamic range of approximately ±200 G is used as the first measuring unit 106 for detecting impact. As a result, the radiation imaging apparatus 103 is able to capture the peak impact at the moment when a significant impact, such as a collision or a fall, is applied.

Here, a case will be considered in which alignment of the radiation imaging apparatus 103 is performed based on an output of the first measuring unit 106. In general, acceleration sensors having a very large dynamic range compared with gravitational acceleration, such as ±200 G, tend to have a larger amount of noise than acceleration sensors having a smaller dynamic range. The amount of noise in the acceleration sensor is about 10 times the resolution. The amount of noise for an acceleration sensor with a dynamic range of ±200 G is about 10 times that for an acceleration sensor with a dynamic range of ±16 G, even for acceleration sensors having the same resolution. For an acceleration sensor having a resolution of 12 bits and a dynamic range of ±200 G, an amount of noise is approximately 1 G.

As described above, when alignment of the radiation imaging apparatus 103 is performed, since the radiation imaging apparatus 103 is not largely moved, a square root of a sum of squares of three-axis outputs of an acceleration sensor is approximately 1 G. For this reason, the first measuring unit 106, which has a dynamic range of ±200 G, is not suitable for fine adjusting the angle or position of the radiation imaging apparatus 103.

Therefore, in the first embodiment, an acceleration sensor having a dynamic range of approximately +16 G is used as the second measuring unit 107 that outputs orientation information. For example, when the resolution is 12 bits, as with the first measuring unit 106, the sensitivity of the second measuring unit 107 is 7.8 mG per 1 LSB (Least Significant Bit). When the output of gravitational acceleration from the acceleration sensor at 0° and 90° is defined as 0 G and +1 G, respectively, then 7.8 mG corresponds to approximately 0.7°, allowing the detection of positional or angular changes of less than 1°.

For example, in skyline imaging of a knee in which it is necessary to perform imaging with the radiation imaging apparatus 103 arranged at a predetermined angle, this function, which enables an angle of the radiation imaging apparatus 103 to be finely adjusted based on an output of an acceleration sensor, is effective. Further, by obtaining angle information of the radiation imaging apparatus 103, the radiation imaging apparatus 103 can be assisted to be oriented to face the radiation generating apparatus 101.

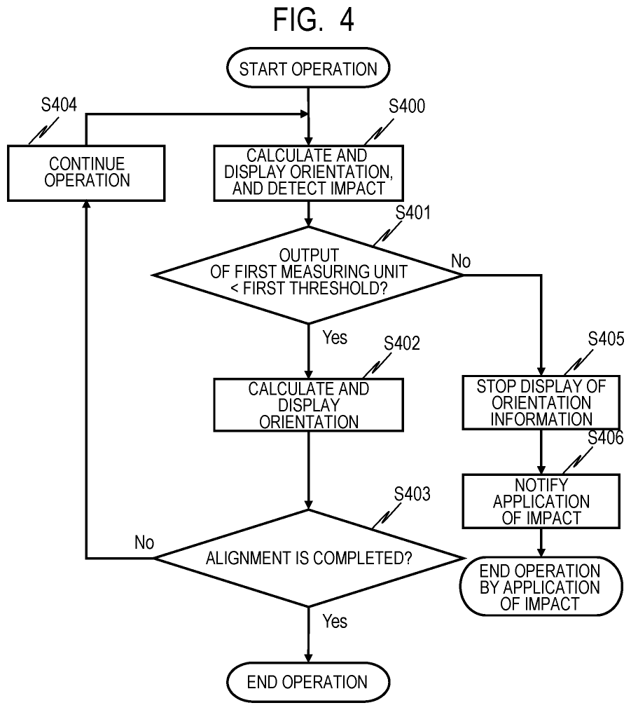

Now, with reference to FIG. 4, a series of operations according to the first embodiment is described. FIG. 4 is a flowchart illustrating a series of operations of the first embodiment. First, when an operation is started and alignment between the radiation generating apparatus 101 and the radiation imaging apparatus 103 is started, in step S400, the controlling unit 105 obtains an output of the second measuring unit 107, and performs calculation and display of orientation information based on the output of the second measuring unit 107. The controlling unit 105 calculates orientation information from an output of the second measuring unit 107 by the above-described method, and transmits the calculated orientation information to the controlling PC 102. The controlling PC 102 causes the received orientation information to be displayed on the display unit 108. Further, the controlling unit 105 obtains an output of the first measuring unit 106 in order to determine application of an impact.

When alignment of the radiation imaging apparatus 103 is performed based on an output of the second measuring unit 107 for orientation detection, the radiation imaging apparatus 103 is not largely moved, as described above. Therefore, a square root of a sum of squares of three-axis outputs of an acceleration sensor is approximately 1 G, and an output substantially equivalent to that of the second measuring unit 107 is also obtained from the first measuring unit 106 for impact detection.

At this time, if an impact is applied to the radiation imaging apparatus 103, outputs of the first measuring unit 106 and the second measuring unit 107 instantaneously change to large values. If an applied impact is on an order of 100 G, since the dynamic range of the second measuring unit 107 is exceeded, an output obtained from the second measuring unit 107 becomes an unreliable value. Therefore, the orientation information of the radiation imaging apparatus 103 calculated based on this value is not suitable for use in alignment.

Thus, the controlling unit 105 performs impact detection using an output of the first measuring unit 106 having a larger dynamic range. In step S401, the controlling unit 105 determines whether an output of the first measuring unit 106 is less than a preset first threshold (a threshold for application of an impact). If it is determined that an output of the first measuring unit 106 is less than the first threshold, the processing proceeds to step S402. It should be noted that the first threshold may be set for an amount of change (a derivative value) of the output value of the first measuring unit 106 instead of the output value of the first measuring unit 106.

In step S402, the controlling unit 105, similarly to step S400, obtains an output of the second measuring unit 107, calculates orientation information using the output of the second measuring unit 107, and transmits the calculated orientation information to the controlling PC 102. The controlling PC 102 causes the received orientation information to be displayed on the display unit 108.

Then, in step S403, the controlling unit 105 determines whether the alignment is completed. Here, the completion of alignment may be determined based on whether the calculated orientation information matches the orientation conditions corresponding to the imaging conditions, or it may be determined in response to an instruction from the operator input via the operating unit 109. It should be noted that an instruction from the operator may be transmitted to the radiation imaging apparatus 103 via the controlling PC 102.

If it is determined in step S403 that the alignment is not completed, the processing proceeds to step S404. In step S404, the controlling unit 105 determines continuation of the operation, and the processing returns to step S400 to continue the alignment. On the other hand, if it is determined in step S403 that alignment is completed, a series of operations is ended. It should be noted that the processing in steps S403 and S404 may be performed by the controlling PC 102.

Further, if it is determined in step S401 that an output of the first measuring unit 106 is equal to or greater than the first threshold, the processing proceeds to step S405. In step S405, the controlling unit 105 transmits, to the controlling PC 102, a stop signal for stopping display of orientation information of the radiation imaging apparatus 103 calculated using an output of the second measuring unit 107. The controlling PC 102, according to the stop signal, causes display of orientation information of the radiation imaging apparatus 103 by the display unit 108 to be stopped.

In step S406, the controlling PC 102 controls the display unit 108 to display a notification to the user in order to prompt the user to perform alignment again because an impact has been applied. After the processing in step S406 is completed, the operation ends. It should be noted that when the operation ends in response to the impact application, the controlling unit 105 may control the radiation detector 104 to prevent erroneous operations, such as image obtainment caused by the impact application. The control may be performed in parallel with the processing in step S405.

Stopping display of orientation information when an impact is applied during alignment is applicable not only to a case in which an angle of the radiation imaging apparatus 103 is displayed, but also to a case in which a position is calculated by time-integrating obtained acceleration data. Also in the case of position calculation, the acceleration output is calculated using saturated values that exceed the dynamic range, so the position information obtained is unreliable. If alignment is continued and imaging is performed in such a state, there is a possibility that a desired image is not obtained and re-imaging is required. Therefore, by monitoring a rapid change in output such as an impact using the first measuring unit 106 and controlling operation of the second measuring unit 107, reliable orientation information can be notified.

As described above, the radiation imaging system 100 according to the first embodiment includes the radiation generating apparatus 101 that emits radiation and the radiation imaging apparatus 103 that performs imaging using the emitted radiation. The radiation imaging apparatus 103 includes the first measuring unit 106 that measures motion information of the radiation imaging apparatus 103, and the second measuring unit 107 that has a dynamic range smaller than a dynamic range of the first measuring unit 106 and measures motion information of the radiation imaging apparatus 103. In the radiation imaging apparatus 103, application of an impact to the radiation imaging apparatus 103 is determined using the first measuring unit 106. Further, in the radiation imaging apparatus 103, information (orientation information) indicating orientation of the radiation imaging apparatus 103 is calculated using the second measuring unit 107.

As described above, the radiation imaging apparatus 103 according to the first embodiment includes the two measuring units having different dynamic ranges. Accordingly, the radiation imaging apparatus 103 can achieve both orientation detection and impact detection.

The radiation imaging apparatus 103 according to the first embodiment may further include the controlling unit 105. The controlling unit 105 can function as an example of a determination unit that determines that an impact has been applied to the radiation imaging apparatus 103 if an output of the first measuring unit 106 or an amount of change of the output of the first measuring unit 106 is equal to or greater than a threshold. Further, the controlling unit 105 may calculate the information indicating the orientation of the radiation imaging apparatus 103 using the second measuring unit 107. The first measuring unit 106 and the second measuring unit 107 may include at least one of an acceleration sensor, a gyro sensor, and an inertial sensor.

Up to this point, a case has been described in which acceleration sensors having different dynamic ranges are used as the first measuring unit 106 that detects an impact of the radiation imaging apparatus 103 and the second measuring unit 107 that measures orientation information of the radiation imaging apparatus 103. In contrast, as characteristics of the first measuring unit 106 and the second measuring unit 107, several characteristics other than a dynamic range may also be considered.

Resolution is given as one example. In the above description, although explanation has been made with the same resolution in order to make a difference in characteristics required for impact detection and orientation detection easy to understand, resolution of the first measuring unit 106 and the second measuring unit 107 is not required to be the same. For example, when resolution of the second measuring unit 107 that measures orientation information is increased, more detailed data is obtained as angle information of the radiation imaging apparatus 103. For example, in a case of the above-described resolution of 12 bits, sensitivity of the acceleration sensor is 7.8 mG per 1 LSB, whereas in a case of a resolution of 13 bits, the sensitivity is 3.9 mG per 1 LSB. Further, in a case of a resolution of 14 bits, the sensitivity is 1.95 mG per 1 LSB. Therefore, acceleration sensors with such resolution may be used to detect finer angle changes.

Therefore, for example, the radiation imaging apparatus 103 may be configured such that resolution of the second measuring unit 107 is higher than resolution of the first measuring unit 106. In this case, alignment accuracy can be improved by obtaining orientation information with finer precision when alignment is performed.

Further, a sampling rate of the first measuring unit 106 and the second measuring unit 107 may be different. As a sampling rate is increased, a data obtainment interval becomes shorter, and therefore, in impact detection, a plot from when an impact is applied to the radiation imaging apparatus 103 to when a peak value is taken can be obtained in more detail. In addition, in the orientation detection, data can be obtained at shorter intervals, so orientation information can be calculated following detailed alignment by the user. In a case where position is calculated by time integration of the acceleration sensor output as illustrated in Equation 4 and Equation 5, the Δt corresponding to the sampling rate is finer, and the accuracy of position calculation is also improved.

Higher sampling rates lead to improved functions of impact detection and orientation detection, but higher sampling rates also lead to higher power consumption. In general, the radiation imaging apparatus 103 is equipped with batteries and can image without an external power supply. However, since battery capacity is limited, an increase in the amount of power supplied to the measuring unit will reduce the number of radiation images that can be captured, which is an inherent function of the radiation imaging apparatus 103. The operation will not be affected if the battery is recharged or replaced when the battery power is low. However, frequent battery replacement will affect the user's operability.

Therefore, there will be described that an operational example to prevent unnecessary increase in power consumption based on the use cases of impact detection and orientation detection. First, with respect to impact detection, since it is not known when an impact is applied to the radiation imaging apparatus 103, it is necessary to always monitor an output of an acceleration sensor. On the other hand, orientation detection is not a function that is used at all times and is used only for a limited period of imaging preparation. Therefore, a sampling rate of the first measuring unit 106 that is always operated in impact detection may be set low, and a sampling rate of the second measuring unit 107 that is used only for a limited period in orientation detection may be set high. For this reason, for example, the radiation imaging apparatus 103 may be configured such that a sampling rate of the first measuring unit 106 is lower than a sampling rate of the second measuring unit 107. In this case, both impact detection and orientation detection can be achieved with limited battery power consumption.

The first embodiment describes an example of using the display unit 108 to notify when a collision is detected. Alternatively, the radiation imaging apparatus 103 may be equipped with a lamp, a display unit, a speaker, or a similar device, and in step S406, the controlling unit 105 may control these devices to notify the user to prompt the user to perform alignment again because an impact has been applied. Such notification may be the lighting of a lamp, display on the display unit, sound or vibration. The controlling unit 105 may also control these lamps, etc. to notify the user that alignment is completed. In this case, the radiation imaging apparatus 103 can notify a user of completion of alignment or detection of an impact, for example, using a lamp, a speaker, or the like (not illustrated), using an output of at least one of the first measuring unit 106 and the second measuring unit 107, and can assist imaging.

### Second Embodiment

As a second embodiment of the present disclosure, a method will be described in which, in a radiation imaging apparatus including two measuring units having different dynamic ranges described in the first embodiment, control of one measuring unit is changed using an output of the other measuring unit. Note that the configuration of the radiation imaging system according to the second embodiment is identical to that of the radiation imaging system 100 according to the first embodiment. Therefore, the same reference numerals are used, and the description is omitted. Hereinafter, a radiation imaging system according to the second embodiment will be described, focusing on differences from the radiation imaging system 100 according to the first embodiment.

In the second embodiment, as in the first embodiment, the radiation imaging apparatus 103 employs an acceleration sensor with a dynamic range of +200 G as the first measuring unit 106, and an acceleration sensor with a dynamic range of +16 G as the second measuring unit 107.

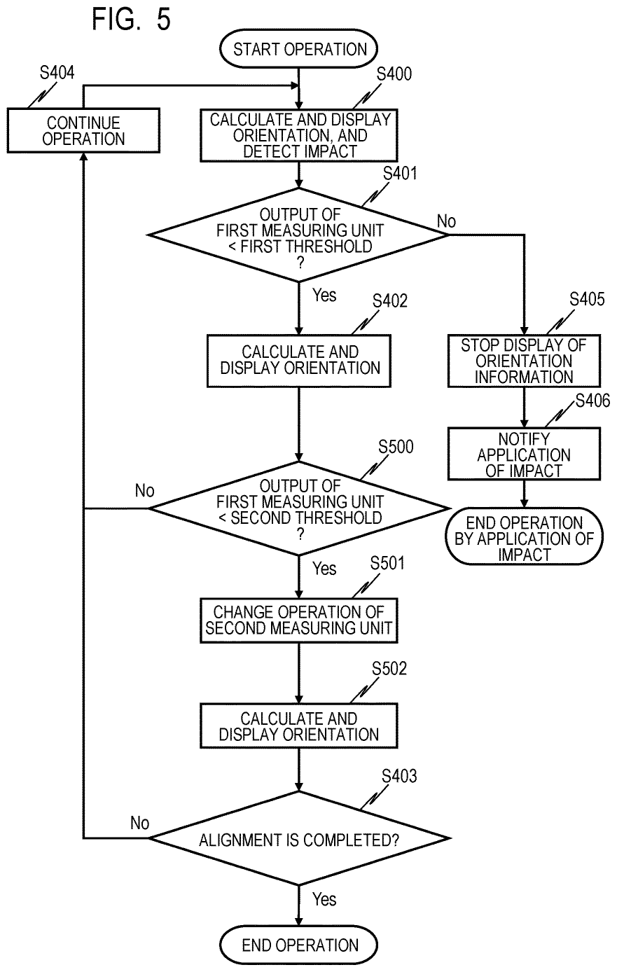

Hereinafter, with reference to FIG. 5, a series of operations according to the second embodiment including processing for switching operation of the second measuring unit 107 based on an output of the first measuring unit 106 will be described. FIG. 5 is a flowchart illustrating a series of operations according to the second embodiment. It should be noted that, in FIG. 5, processing similar to processing in the series of operations according to the first embodiment is denoted by the same reference numerals, and description thereof is omitted. In the series of operations according to the second embodiment, when calculation and display of orientation information are performed in step S402, the processing proceeds to step S500.

Hereinafter, a method for improving measurement accuracy of the second measuring unit 107 based on an output of the first measuring unit 106 will be described. The output of the acceleration sensor during alignment is about 1 G, which is the square root of the sum of the squares of the three-axis outputs, as mentioned above. This is common regardless of the dynamic range of the acceleration sensor. In the second embodiment, an acceleration sensor with a dynamic range of +16 G is used as the second measuring unit 107, but in general, the dynamic range of acceleration sensors can often be changed by settings. For example, if the second measuring unit 107 is an acceleration sensor capable of having a dynamic range set in a range from +1 G to +16 G, if an output exceeds the dynamic range as described above, accuracy of orientation information is lost.

In contrast, if an output can be expected as in fine adjustment of an angle, a dynamic range can be reduced, and resolution per 1 LSB can be improved. In the second embodiment, in step S500, the controlling unit 105 determines whether an output of the first measuring unit 106 different from the second measuring unit 107 for orientation detection is less than a preset second threshold. Here, the second threshold is a threshold of an output value during alignment in the first measuring unit 106. If it is determined that an output of the first measuring unit 106 is equal to or greater than the second threshold, the processing proceeds to step S404, and operation is continued. On the other hand, if it is determined that an output of the first measuring unit 106 is less than the second threshold, the processing proceeds to step S501. It should be noted that the second threshold may be set for an amount of change (a derivative value) of the output value of the first measuring unit 106 instead of the output value of the first measuring unit 106.

In step S501, the controlling unit 105 reduces a dynamic range of the second measuring unit 107 for orientation detection so that resolution per 1 LSB can be improved. Further, the controlling unit 105 can increase a sampling rate of the second measuring unit 107 to improve time resolution, or can change resolution (number of bits) itself. In such a case, the second measuring unit 107 can detect, in detail, a subtle change in an angle of alignment and a change in position information due to time integration of an acceleration output. Such a change of operation of the second measuring unit 107 is one example, and an item to be changed may be set in advance, or may be selected by a user each time.

After the controlling unit 105 changes operation in step S501, the controlling unit 105 obtains an output of the second measuring unit 107 in step S502, and performs orientation calculation and display of orientation information. Orientation calculation and display of orientation information in step S502 may be performed similarly to calculation and display of orientation information in step S400 and step S402. Then, in step S403, the controlling unit 105 determines whether the alignment is completed. Since the subsequent processing is the same as that described in the first embodiment, the description is omitted.

In the second embodiment, a method has been described in which operation of the second measuring unit 107 is changed based on an output of the first measuring unit 106 to realize orientation detection with high accuracy. In contrast, it is also possible to realize impact detection with high accuracy by changing operation of the first measuring unit 106 based on an output of the second measuring unit 107.

As described in the first embodiment, generally, a battery is mounted in the radiation imaging apparatus 103, and if a sampling rate is increased more than necessary, power consumption increases and an influence is exerted on operability of a user. The first measuring unit 106 for impact detection needs to be always operated in preparation for an impact that is not known when application occurs. However, in order to suppress an influence on battery capacity described above, it is appropriate that a sampling rate of the first measuring unit 106 is set lower than a sampling rate of the second measuring unit 107 whose operation is limited.

On the other hand, if a sampling rate of the first measuring unit 106 is excessively reduced, there is a possibility that a sampling interval becomes coarse and a peak of an impact cannot be captured. Therefore, the controlling unit 105 can change a sampling rate of the first measuring unit 106 to a high value when an output of the second measuring unit 107 becomes equal to or greater than a preset third threshold (a threshold for movement detection in the second measuring unit 107). Accordingly, by changing operation of the first measuring unit 106 in response to a change in an output of the second measuring unit 107 due to movement of the radiation imaging apparatus 103, a peak of an impact can be more easily detected.

In other words, using the second measuring unit 107, it is possible to detect a situation in which an impact is likely to be applied to the radiation imaging apparatus 103, for example, a situation in which the radiation imaging apparatus 103 is moved for alignment or the like, and to change operation of the first measuring unit 106. In such a case, by changing a sampling rate of the first measuring unit 106 to a high value according to a situation in which an impact is likely to be applied to the radiation imaging apparatus 103, a peak of an impact can be more easily detected. It should be noted that the third threshold may be set for an amount of change (a derivative value) of an output value of the second measuring unit 107 instead of the output value of the second measuring unit 107.

It should be noted that such processing may be performed at all times when power of the radiation imaging apparatus 103 is turned on. Further, while operation involving movement of the radiation imaging apparatus 103 such as a series of operations illustrated in FIG. 5 is continuously performed, the controlling unit 105 may temporarily stop such processing for reduction of processing load and may maintain the changed operation of the measuring unit.

As described above, in the radiation imaging apparatus 103 according to the second embodiment, operation of one of the first measuring unit 106 and the second measuring unit 107 is changed based on an output of the other. For example, the controlling unit 105 may perform at least one of reducing a dynamic range, increasing resolution, and increasing a sampling rate of the second measuring unit 107 if an output of the output of the first measuring unit 106 or an amount of change of the output of the first measuring unit 106 is less than a threshold. Further, the controlling unit 105 may, for example, increase a sampling rate of the first measuring unit 106 when an output of the second measuring unit 107 or an amount of change of the output of the second measuring unit 107 is equal to or greater than a threshold.

With such a configuration, the radiation imaging apparatus 103 according to the second embodiment can change operation of one of the first measuring unit 106 for impact detection and the second measuring unit 107 for orientation detection having different dynamic ranges based on an output of the other. For this reason, the radiation imaging apparatus 103 can improve both functions of orientation detection and impact detection.

It should be noted that, in the second embodiment, a case has been described in which the controlling unit 105 changes operation of one measuring unit using an output of the other measuring unit. In contrast, the controlling unit 105 may change operation of the one measuring unit using an output of the one measuring unit. For example, the controlling unit 105 may, if an output of the second measuring unit 107 or an amount of change of the output of the second measuring unit 107 is less than a predetermined threshold, reduce a dynamic range of the second measuring unit 107, increase resolution, or increase a sampling rate. Further, the controlling unit 105 may, if an output of the first measuring unit 106 or an amount of change of the output of the first measuring unit 106 is less than a predetermined threshold, reduce a sampling rate of the first measuring unit 106. In these cases, the accuracy of orientation detection and impact detection can still be improved. It should be noted that, if change of operation is determined using an output of the second measuring unit 107 having a small dynamic range, processing for changing operation can be performed according to a change in a finer output value, and accuracy of change of operation can be improved.

### Third Embodiment

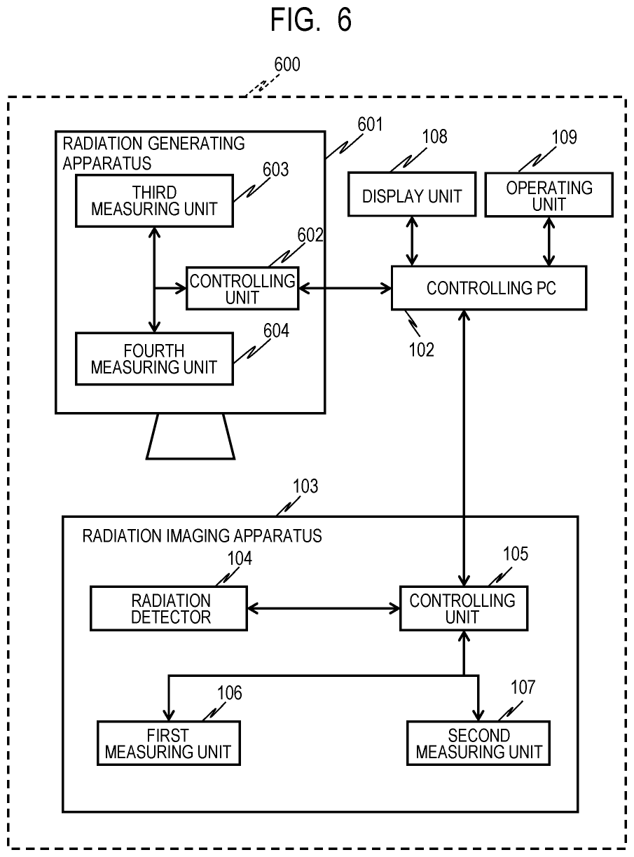

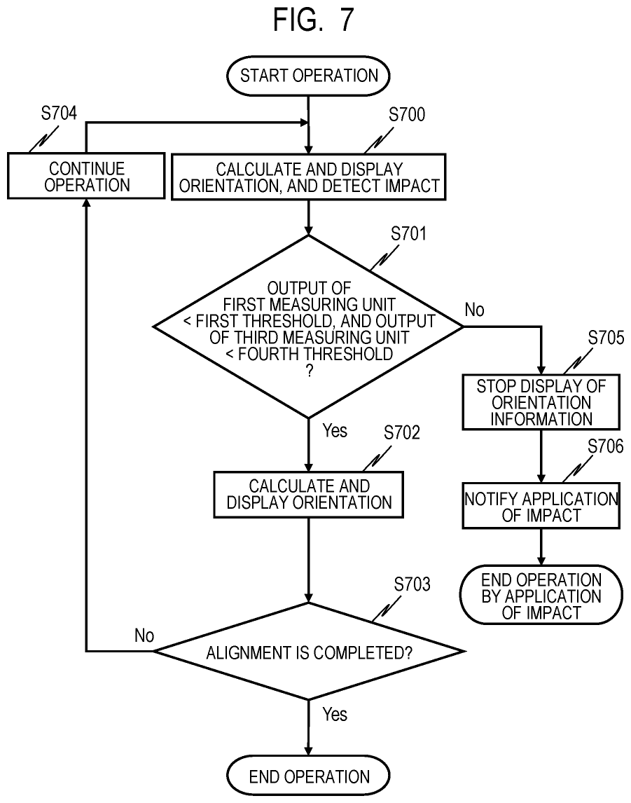

Hereinafter, with reference to FIG. 6 and FIG. 7, a radiation imaging system, a radiation generating apparatus, and a method of operating a radiation generating apparatus according to a third embodiment of the present disclosure will be described. In the third embodiment, in addition to a radiation imaging apparatus including two measuring units having different dynamic ranges as measuring units, a radiation imaging system having a radiation generating apparatus including two measuring units having different dynamic ranges as measuring units is described as an example. Also in the third embodiment, both orientation detection and impact detection can be achieved by the radiation imaging system. FIG. 6 is a configuration diagram of a radiation imaging system 600 in the third embodiment.

It should be noted that, with respect to a configuration of the radiation imaging system 600 according to the third embodiment, description is omitted by using the same reference numerals for a configuration similar to a configuration of the radiation imaging system 100 according to the first embodiment. For example, with respect to the radiation imaging apparatus 103 including two measuring units having different dynamic ranges as measuring units, description is omitted since it has been described in the first embodiment and the second embodiment. Hereinafter, the radiation imaging system 600 according to the third embodiment will be described, focusing on differences from the radiation imaging system 100 according to the first embodiment. The radiation generating apparatus 601 according to the third embodiment includes a controlling unit 602, a third measuring unit 603, and a fourth measuring unit 604.

The controlling unit 602 can control operation of a radiation generator such as a radiation tube that generates radiation based on a signal from the controlling PC 102 or the like. Further, the controlling unit 602 calculates orientation information of the radiation generating apparatus 601 based on an output of the fourth measuring unit 604. Further, the controlling unit 602 can determine an impact based on an output of the third measuring unit 603. The controlling unit 602 can be configured using any processor. Further, each function of the controlling unit 602 may be realized by a processor such as CPU or MPU executing a software module stored in a memory or other storage device. It should be noted that the processor may be a GPU, an FPGA or the like, for example. Each function may be configured by a circuit or other device that performs a specific function, such as an ASIC.

The third measuring unit 603 and the fourth measuring unit 604 are configured using two acceleration sensors having different dynamic ranges, similarly to the above-described radiation imaging apparatus 103. In the third embodiment, an acceleration sensor having a dynamic range of ±200 G is used as the third measuring unit 603. Further, an acceleration sensor having a dynamic range of ±16 G is used as the fourth measuring unit 604. The third measuring unit 603 and the fourth measuring unit 604 are connected to the controlling PC 102 via the controlling unit 602 of the radiation generating apparatus 601. The third measuring unit 603 is used to detect an impact applied to the radiation generating apparatus 601, and the fourth measuring unit 604 is used for an angle adjustment and alignment of the radiation generating apparatus 601. It should be noted that, since a method of impact detection and a method of alignment are the same as the method of impact detection and the method of alignment in the first embodiment, description is omitted. In the third embodiment, by including two measuring units having different dynamic ranges in the radiation generating apparatus 601, both orientation detection and impact detection of the radiation generating apparatus 601 can be achieved, similarly to the orientation detection and impact detection of the radiation imaging apparatus 103 according to the first embodiment.

The radiation generating apparatus 601 is attached to a movable rail fixed to a floor, a wall surface or a ceiling of an examination room, and is capable of movement in three-dimensional directions of X, Y and Z axes and rotation with respect to an irradiation direction of radiation according to operation of a user. A user moves the radiation generating apparatus 601 and the radiation imaging apparatus 103, and performs imaging after confirming that arrangement corresponding to an imaging technique is achieved. At that time, similarly to that there is a possibility that an unexpected impact due to a fall or a collision is applied to the radiation imaging apparatus 103 as described in the first embodiment, there is a possibility that an impact is also applied to the radiation generating apparatus 601. Generally, since the radiation generating apparatus 601 is larger and heavier than the radiation imaging apparatus 103, for example, it is often moved only in a state in which an interlock is released by pressing a switch. Here, if a switch is released during movement, a lock is applied and movement of the radiation generating apparatus 601 stops. When a state in which the radiation generating apparatus 601 is moving changes to a state in which the radiation generating apparatus 601 is rapidly stopped, acceleration output from the third measuring unit 603 mounted on the radiation generating apparatus 601 changes. Further, an impact may also be applied to the radiation generating apparatus 601 by colliding with surrounding equipment during movement of the radiation generating apparatus 601. In these cases, the controlling unit 602 can detect that an impact has been applied to the radiation generating apparatus 601 based on an output from the third measuring unit 603.

For example, when an output of the fourth measuring unit 604 is time-integrated to calculate a movement amount of the radiation generating apparatus 601 for alignment or the like, if an impact is applied and exceeds a dynamic range of the fourth measuring unit 604, an accurate movement amount cannot be calculated. Therefore, if it is recognized that an output of the third measuring unit 603 exceeds a preset fourth threshold and an impact has been applied to the radiation generating apparatus 601, display of orientation information of the radiation generating apparatus 601, such as an angle and a movement amount calculated based on an output of the fourth measuring unit 604, is stopped. At this time, in addition to stopping display of orientation information, it is also possible to notify a user by, for example, causing display of that an impact has been applied to the radiation generating apparatus 601 to be displayed on the display unit 108.

Now, with reference to FIG. 7, a series of operations according to the third embodiment is described. A series of operations according to the third embodiment is the same as the series of operations according to the first embodiment except that operation in the radiation generating apparatus 601 is similarly performed in each processing.

First, when operation is started and alignment of the radiation generating apparatus 601 and the radiation imaging apparatus 103 is started, the processing proceeds to step S700. In step S700, the controlling unit 105 obtains an output of the second measuring unit 107, calculates orientation information of the radiation imaging apparatus 103 from the output of the second measuring unit 107, and transmits the calculated orientation information to the controlling PC 102. The controlling PC 102 causes the received orientation information of the radiation imaging apparatus 103 to be displayed on the display unit 108. Further, the controlling unit 105 obtains an output of the first measuring unit 106 in order to determine application of an impact to the radiation imaging apparatus 103. On the other hand, the controlling unit 602 obtains an output of the fourth measuring unit 604, calculates orientation information of the radiation generating apparatus 601 from the output of the fourth measuring unit 604, and transmits the calculated orientation information to the controlling PC 102. The controlling PC 102 causes the received orientation information of the radiation generating apparatus 601 to be displayed on the display unit 108. Further, the controlling unit 602 obtains an output of the third measuring unit 603 in order to determine application of an impact to the radiation generating apparatus 601. It should be noted that a calculation method of orientation information may be the same as a calculation method described in the first embodiment.

In step S701, the controlling unit 105 determines whether an output of the first measuring unit 106 is less than a preset first threshold (a threshold for application of an impact). Further, the controlling unit 602 determines whether an output of the third measuring unit 603 is less than a preset fourth threshold (a threshold for application of an impact). If it is determined that an output of the first measuring unit 106 is less than the first threshold and that an output of the third measuring unit 603 is less than the fourth threshold, the processing proceeds to step S702. It should be noted that the fourth threshold may be set for an amount of change (a derivative value) of an output value of the third measuring unit 603 instead of the output value of the third measuring unit 603.

In step S702, similarly to step S700, the controlling unit 105 obtains an output of the second measuring unit 107, calculates orientation information of the radiation imaging apparatus 103 using the output of the second measuring unit 107, and transmits the calculated orientation information to the controlling PC 102. The controlling PC 102 causes the received orientation information to be displayed on the display unit 108. Further, the controlling unit 602 obtains an output of the fourth measuring unit 604, calculates orientation information of the radiation generating apparatus 601 from the output of the fourth measuring unit 604, and transmits the calculated orientation information to the controlling PC 102. The controlling PC 102 causes the received orientation information of the radiation generating apparatus 601 to be displayed on the display unit 108.

Then, in step S703, the controlling unit 105 and the controlling unit 602 determine whether alignment is completed. Here, the completion of alignment may be determined based on whether the calculated orientation information matches the orientation conditions corresponding to the imaging conditions, or it may be determined in response to an instruction from the operator entered via the operating unit 109. It should be noted that an instruction from an operator may be transmitted to the radiation imaging apparatus 103 and the radiation generating apparatus 601 via the controlling PC 102.

If it is determined in step S703 that the alignment is not completed, the processing proceeds to step S704. In step S704, continuation of operation is determined by the controlling unit 105 and the controlling unit 602, and the processing returns to step S700 to continue alignment. On the other hand, when it is determined in step S703 that alignment is completed, a series of operations is ended. It should be noted that the processing in steps S703 and S704 may be performed by the controlling PC 102.

Further, in step S701, if it is determined that an output of the first measuring unit 106 is equal to or greater than the first threshold or that an output of the third measuring unit 603 is equal to or greater than the fourth threshold, the processing proceeds to step S705. In step S705, if an output of the first measuring unit 106 is equal to or greater than the first threshold, the controlling unit 105 transmits, to the controlling PC 102, a stop signal for stopping display of orientation information of the radiation imaging apparatus 103 calculated using an output of the second measuring unit 107. The controlling PC 102, according to the stop signal, causes display of orientation information of the radiation imaging apparatus 103 by the display unit 108 to be stopped. On the other hand, if an output of the third measuring unit 603 is equal to or greater than the fourth threshold, the controlling unit 602 transmits, to the controlling PC 102, a stop signal for stopping display of orientation information of the radiation generating apparatus 601 calculated using an output of the fourth measuring unit 604. The controlling PC 102, according to the stop signal, causes display of orientation information of the radiation generating apparatus 601 by the display unit 108 to be stopped.

In step S706, the controlling PC 102 controls the display unit 108 to display a notification to the user in order to prompt the user to perform alignment again because an impact has been applied. When processing in step S706 is completed, operation of the radiation imaging apparatus 103 and the radiation generating apparatus 601 is ended according to application of an impact. It should be noted that when the operation ends in response to the impact application, the controlling unit 105 may control the radiation detector 104 to prevent erroneous operations, such as image obtainment caused by the impact application. Further, the controlling unit 602 may control the radiation generating apparatus 601 so as to prevent erroneous operation such as radiation irradiation caused by application of an impact. The control may be performed in parallel with the processing in step S705.

As described above, the radiation imaging system 600 according to the third embodiment includes the radiation generating apparatus 601 that emits radiation and the radiation imaging apparatus 103 that performs imaging using the emitted radiation. The radiation generating apparatus 601 includes a third measuring unit 603 that measures motion information of the radiation generating apparatus 601, and a fourth measuring unit 604 that has a dynamic range smaller than a dynamic range of the third measuring unit 603 and measures motion information of the radiation generating apparatus 601. In the radiation generating apparatus 601, application of an impact to the radiation generating apparatus 601 is determined using the third measuring unit 603. Further, in the radiation generating apparatus 601, information (orientation information) indicating orientation of the radiation generating apparatus 601 is calculated using the fourth measuring unit 604.

As described above, the radiation generating apparatus 601 according to the third embodiment includes the two measuring units having different dynamic ranges. Accordingly, the radiation generating apparatus 601 can achieve both orientation detection and impact detection. Further, both orientation information of the radiation generating apparatus 601 and the radiation imaging apparatus 103 obtained from the second measuring unit 107 mounted on the radiation imaging apparatus 103 and the fourth measuring unit 604 mounted on the radiation generating apparatus 601 can be combined and used for alignment. Accordingly, the radiation imaging system 600 can assist alignment of the radiation generating apparatus 601 and the radiation imaging apparatus 103.

It should be noted that the radiation imaging system 600 according to the third embodiment further includes the controlling PC 102. The controlling PC 102 can function as an example of a control apparatus that controls the radiation imaging apparatus 103 and the radiation generating apparatus 601. Further, the controlling PC 102 can function as an example of a display controlling unit that causes the display unit 108 to display at least one of a determination result of application of an impact to the radiation imaging apparatus 103, the information indicating the orientation of the radiation imaging apparatus 103, a determination result of application of an impact to the radiation generating apparatus 601, and the information indicating the orientation of the radiation generating apparatus 601. In this case, the radiation imaging system 600 can, for example, cause the display unit 108 to display a screen as illustrated in FIG. 3A and FIG. 3B, and can further assist alignment of the radiation imaging apparatus 103 and the radiation generating apparatus 601 by a user. It should be noted that the controlling PC 102 may control display of the display unit 108 according to a signal from at least one of the controlling unit 105 and the controlling unit 602. In this case, at least one of the controlling unit 105 and the controlling unit 602 may function as an example of a display controlling unit.

The radiation generating apparatus 601 according to the third embodiment further includes the controlling unit 602. The controlling unit 602 can function as an example of a determination unit that determines that an impact has been applied to the radiation generating apparatus 601 if an output of the third measuring unit 603 or an amount of change of the output of the third measuring unit 603 is equal to or greater than a threshold. Further, the controlling unit 602 can calculate information indicating the orientation of the radiation generating apparatus 601 using the fourth measuring unit 604. Further, the third measuring unit 603 and the fourth measuring unit 604 can include at least one of an acceleration sensor, a gyro sensor, and an inertial sensor.

Further, also for the radiation generating apparatus 601, similarly to the first embodiment, resolution and a sampling rate may be made different between the third measuring unit 603 and the fourth measuring unit 604. For example, the radiation generating apparatus 601 may be configured such that resolution of the fourth measuring unit 604 is higher than resolution of the third measuring unit 603. In this case, orientation information for performing alignment can be obtained with finer accuracy, and accuracy of alignment can be improved. Further, for example, the radiation generating apparatus 601 may be configured such that a sampling rate of the third measuring unit 603 is lower than a sampling rate of the fourth measuring unit 604. In this case, both impact detection and orientation detection can be achieved with limited battery power consumption.

It should be noted that, in the third embodiment, the controlling unit 105 functions as a determination unit that performs determination of collision of the radiation imaging apparatus 103, and the controlling unit 602 functions as a determination unit that performs determination of collision of the radiation generating apparatus 601. In contrast, outputs of the first measuring unit 106 and the third measuring unit 603 may be transmitted to the controlling PC 102, and determination of collision of the radiation imaging apparatus 103 and collision of the radiation generating apparatus 601 may be performed by the controlling PC 102. In this case, the controlling PC 102 can function as a determination unit that performs determination of collision of the radiation imaging apparatus 103 and collision of the radiation generating apparatus 601.

Further, an output of the first measuring unit 106 may be transmitted to the radiation generating apparatus 601, and determination of collision of the radiation imaging apparatus 103 and collision of the radiation generating apparatus 601 may be performed by the controlling unit 602. In this case, the controlling unit 602 can function as a determination unit that performs determination of collision of the radiation imaging apparatus 103 and collision of the radiation generating apparatus 601. Similarly, an output of the third measuring unit 603 may be transmitted to the radiation imaging apparatus 103, and determination of collision of the radiation imaging apparatus 103 and collision of the radiation generating apparatus 601 may be performed by the controlling unit 105. In this case, the controlling unit 105 can function as a determination unit that performs determination of collision of the radiation imaging apparatus 103 and collision of the radiation generating apparatus 601. It should be noted that communication between the radiation imaging apparatus 103 and the radiation generating apparatus 601 may be performed via the controlling PC 102 or may be performed directly.

Further, also for the radiation generating apparatus 601 according to the third embodiment, processing for changing operation of one measuring unit using an output of the other measuring unit described in the second embodiment may be applied. More specifically, in the radiation generating apparatus 601, operation of one of the third measuring unit 603 and the fourth measuring unit 604 may be changed based on an output of the other. For example, if an output of the third measuring unit 603 or an amount of change of the output of the third measuring unit 603 is less than a preset fifth threshold, the controlling unit 602 may determine that alignment of the radiation generating apparatus 601 is being performed, and change a dynamic range of the fourth measuring unit 604 to be smaller. Further, in a similar situation, the controlling unit 602 may increase a sampling rate of the fourth measuring unit 604 or increase resolution. Therefore, the controlling unit 602 may perform at least one of reducing a dynamic range of the fourth measuring unit 604, increasing resolution, and increasing a sampling rate when an output of the third measuring unit 603 or an amount of change of the output of the third measuring unit 603 is less than a threshold. In such a case, detailed orientation information of the radiation generating apparatus 601 can be calculated, and assistance can be provided for a user to arrange the radiation generating apparatus 601 at a predetermined position.

Further, the controlling unit 602 may increase a sampling rate of the third measuring unit 603 if an output of the fourth measuring unit 604 or an amount of change of the output of the fourth measuring unit 604 is equal to or greater than a threshold. Accordingly, by changing operation of the third measuring unit 603 in response to a change in an output of the fourth measuring unit 604 due to movement of the radiation generating apparatus 601, a peak of an impact can be more easily detected. As described above, by applying processing according to the second embodiment to the radiation generating apparatus 601 according to the third embodiment, the radiation generating apparatus 601 can improve both functions of orientation detection and impact detection.

Further, the controlling unit 602 may change operation of one measuring unit using an output of the one measuring unit. For example, the controlling unit 602 may, if an output of the fourth measuring unit 604 or an amount of change of the output of the fourth measuring unit 604 is less than a predetermined threshold, reduce a dynamic range of the fourth measuring unit 604. Further, the controlling unit 602 may, if an output of the third measuring unit 603 or an amount of change of the output of the third measuring unit 603 is less than a predetermined threshold, reduce a sampling rate of the third measuring unit 603. In these cases, the accuracy of orientation detection and impact detection can be improved. It should be noted that, if change of operation is determined using an output of the fourth measuring unit 604 having a small dynamic range, processing for changing operation can be performed according to a change in a finer output, and accuracy of change of operation can be improved.

The third embodiment describes an example of using the display unit 108 to notify when a collision is detected. Alternatively, the radiation imaging apparatus 103 may be equipped with a lamp, a display unit, a speaker, or a similar device, and in step S706, the controlling unit 105 may control these devices to notify the user to prompt the user to perform alignment again because an impact has been applied. Similarly, the radiation generating apparatus 601 may be equipped with a lamp, a display unit, a speaker, or a similar device, and in step S706, the controlling unit 602 may control these devices to notify the user to prompt the user to perform alignment again because an impact has been applied. Such notification may be the lighting of a lamp, display on the display unit, sound or vibration. The controlling unit 105 and the controlling unit 602 may also control these lamps, etc. to notify the user that alignment is completed.

In this case, the radiation imaging apparatus 103 can notify a user of completion of alignment or detection of an impact, for example, using a lamp, a speaker, or the like (not illustrated) using an output of at least one of the first measuring unit 106 and the second measuring unit 107, and can assist imaging. Further, the radiation generating apparatus 601 can notify a user of completion of alignment or detection of an impact, for example, using a lamp, a speaker, or the like (not illustrated) using an output of at least one of the third measuring unit 603 and the fourth measuring unit 604, and can assist imaging.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

Here, the processor or circuit may include a central processing unit (CPU), a micro processing unit (MPU), a graphics processing unit (GPU), an application specific integrated circuit (ASIC), or a field-programmable gate array (FPGA). In addition, the processor or circuit may include a digital signal processor (DSP), a data flow processor (DFP), or a neural processing unit (NPU).

According to at least one embodiment of the present disclosure, both functions of orientation detection and impact detection of the radiation imaging apparatus can be improved.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A radiation imaging apparatus, comprising:
a first measuring unit configured to measure motion information of the radiation imaging apparatus; and
a second measuring unit having a dynamic range smaller than a dynamic range of the first measuring unit and configured to measure motion information of the radiation imaging apparatus,
wherein:
application of an impact to the radiation imaging apparatus is determined using the first measuring unit; and
information indicating an orientation of the radiation imaging apparatus is calculated using the second measuring unit.

2. The radiation imaging apparatus according to claim 1, further comprising a determination unit configured to determine that an impact has been applied to the radiation imaging apparatus if an output of the first measuring unit or an amount of change of the output of the first measuring unit is equal to or greater than a threshold.

3. The radiation imaging apparatus according to claim 1, wherein imaging is assisted using an output of at least one of the first measuring unit and the second measuring unit.

4. The radiation imaging apparatus according to claim 1, wherein based on an output of one of the first measuring unit and the second measuring unit, an operation of the other is changed.

5. The radiation imaging apparatus according to claim 4, further comprising a controlling unit configured to perform at least one of reducing a dynamic range, increasing a resolution, and increasing a sampling rate of the second measuring unit if an output of the first measuring unit or an amount of change of the output of the first measuring unit is less than a threshold.

6. The radiation imaging apparatus according to claim 4, further comprising a controlling unit configured to increase a sampling rate of the first measuring unit if an output of the second measuring unit or an amount of change of the output of the second measuring unit is equal to or greater than a threshold.

7. The radiation imaging apparatus according to claim 1, wherein a resolution of the second measuring unit is higher than a resolution of the first measuring unit.

8. The radiation imaging apparatus according to claim 1, wherein a sampling rate of the first measuring unit is lower than a sampling rate of the second measuring unit.

9. The radiation imaging apparatus according to claim 1, wherein the first measuring unit and the second measuring unit include at least one of an acceleration sensor, a gyro sensor, and an inertial sensor.

10. A radiation imaging system, comprising:
a radiation generating apparatus arranged to emit radiation; and
the radiation imaging apparatus according to claim 1 configured to perform imaging with the emitted radiation.

11. The radiation imaging system according to claim 10, further comprising a display controlling unit configured to cause a display unit to display at least one of a determination result of application of an impact to the radiation imaging apparatus, information indicating an orientation of the radiation imaging apparatus, a determination result of application of an impact to the radiation generating apparatus, and information indicating an orientation of the radiation generating apparatus.

12. The radiation imaging system according to claim 11,
wherein the display controlling unit:
causes the display unit to display a determination result of application of an impact to the radiation imaging apparatus and does not cause the display unit to display information indicating an orientation of the radiation imaging apparatus if an output of the first measuring unit or an amount of change of the output of the first measuring unit is equal to or greater than a threshold; and
does not cause the display unit not to display a determination result of application of an impact to the radiation imaging apparatus and causes the display unit to display information indicating an orientation of the radiation imaging apparatus if the output of the first measuring unit or the amount of change of the output of the first measuring unit is less than a threshold.

13. A method of operating a radiation imaging apparatus comprising a first measuring unit configured to measure motion information of the radiation imaging apparatus, and a second measuring unit having a dynamic range smaller than a dynamic range of the first measuring unit and configured to measure motion information of the radiation imaging apparatus, the method comprising:
determining application of an impact to the radiation imaging apparatus using the first measuring unit; and
calculating information indicating an orientation of the radiation imaging apparatus using the second measuring unit.

14. A non-transitory computer-readable storage medium storing a program that, when executed by a processor, causes the processor to execute respective steps of the method of operating the radiation imaging apparatus according to claim 13.
